(19) United States
(12) Patent Application Publication
(10) Pub. No.: US 2022/0096467 A1
(43) Pub. Date: Mar. 31, 2022

(54) COMBINATION OF SELECTIVE ALPHA-ADRENERGIC RECEPTOR AGONIST OR AN ANTICHOLINERGIC AGENT AND LIPOIC ACID AND USES THEREOF

(71) Applicant: Cellix Bio Private Limited, Hyderabad (IN)

(72) Inventor: Mahesh Kandula, East Godavari District, Andhra Pradesh (IN)

(73) Assignee: Cellix Bio Provate Limited, Hyderabad (IN)

(21) Appl. No.: 17/310,032

(22) PCT Filed: Jan. 3, 2020
(86) PCT No.: PCT/IB2020/050028

§ 371 Date: Jul. 12, 2021

(30) Foreign Application Priority Data
Jan. 12, 2019 (IN) 201941001491
Feb. 5, 2019 (IB) PCT/IB2019/050901

(51) Int. Cl. A61K 31/498; A61K 31/385; A61K 31/4174

(52) U.S. Cl. CPC A61K 31/498; A61K 31/4174; A61K 31/385

(57) ABSTRACT

The present disclosure relates to pharmaceutical compositions comprising a therapeutically effective amount of a selective alpha-adrenergic receptor agonist or an anticholinergic agentor a pharmaceutically acceptable salt or a stereoisomer thereof in combination with a therapeutically effective amount of lipoic acid or a pharmaceutically acceptable salt or a stereoisomer thereof; wherein the selective alpha-adrenergic receptor agonist or anticholinergic agent is selected from the group consisting of pilocarpine, brimonidine and oxymetazoline, or a pharmaceutically acceptable salt or a stereoisomer thereof, and their uses in the treatment or alleviation of xerostomia, dermal diseases and eye disorders.

### PRIORITY

The present application claims the benefit of the Indian Provisional Application No. 201941001491 filed on 12 Jan. 2019 and International Application no. PCT/IB2019/050901 filed on 5 Feb. 2019, the entire disclosures of which are relied on for all purpose and are incorporated into this application by reference.

### TECHNICAL FIELD OF THE INVENTION

The present invention relates to pharmaceutical compositions and methods of using the same for the treatment of or alleviation of burning or xerostomia particularly of the oral cavity burning mouth syndrome and eye diseases or disorders.

### BACKGROUND OF THE INVENTION

Xerostomia, also known as dry mouth, is a condition in which an excessive dryness within the oral cavity takes place, due to insufficient salivary production. Xerostomia is not a disease itself but is a side effect of a radiation to the head and neck, or a side effect of a wide variety of medications. Few common problems associated with xerostomia include but are not limited to constant sore throat, burning sensations, difficulty speaking and swallowing and dry nasal passages, all related to the decreased level of fluids in the oral cavity.

Systemic pharmacological treatments include parasympathomimetic agents such as pilocarpine, cevimeline and bethanechol that act on β-adrenergic receptors and stimulate secretion from salivary glands. In clinical practice, they are used to treat xerostomia after radiotherapy for head and neck cancer but are associated with side effects such as headache and sweating.

Xerostomia remains an unresolved common complaint especially among the geriatric population, despite seeking medical or dental consultation. Managing acute pathology often relies on the addressing underlying pathology and symptoms of the disease. There is currently a need in the art for new compositions for treating or delaying the onset of xerostomia and its associated complications progression.

The supplement alpha lipoic acid (ALA) showed some promise for helping glaucoma patients. Patients who had open angle glaucoma were given the 150 mg of ALA per day to see if the supplement made a difference to the effects of this common eye disease. The study showed that 45-47% of the eyes in the study enhanced color visual fields and visual sensitivity versus the controls. The controls only received topical medical therapy. Alpha lipoic acid is present in every cell in the human body. It converts glucose into energy.

Age-related eye diseases, in many cases are not sudden but tend to develop slowly as a person ages. Of many age-related eye diseases, there are four major diseases that are recognized, which can be detected and treated if a comprehensive eye examination is performed. These four age-related eye diseases—Macular Degeneration, Cataract, Glaucoma and Diabetic Retinopathy are expected to dramatically increase if left untreated and can cause vision loss and blindness. Population that is most at risk for developing eye disease is unaware of the factors that make them susceptible. As the proportion of the elderly population increases around the world, the prevalence and effects of age-related eye diseases are also increasing. The leading causes of blindness and low vision are primarily age-related eye diseases such as age-related macular degeneration, cataract, diabetic retinopathy, and glaucoma. Age-related cataract will become an even larger percentage of the causes of blindness worldwide, and glaucoma and age-related macular degeneration will emerge as public health issues.

Most common eye problems include Refractive errors, Cataracts—clouded lenses, Optic nerve disorders, including glaucoma, Retinal disorders—problems with the nerve layer at the back of the eye, Macular degeneration—a disease that destroys sharp, central vision, Diabetic eye problems and Conjunctivitis—an infection also known as pinkeye. Presbyopia is progressive loss of accommodation resulting in loss of visual ability to focus on objects located at different distances. Accommodation in humans is performed by ciliary muscle and iris sphincter contractions, convergence and changes in the shape and position of the lens. The latter action is passive, meaning the lens changes are dependent on the ciliary muscle and iris contractions. Also, when the centre of the accommodation is active, the ciliary muscle contraction is stimulated and miosis and convergence occurs in normal binocular patients.

Lipoic acid is a fatty acid that is in most foods, but only in very tiny amounts. Certain organ meats, broccoli, yeast extract and spinach have slightly higher quantities of lipoic acid, but not a significant amount. Lipoic acid found in foods is not readily usable by the body.

### SUMMARY OF THE INVENTION

The present disclosure relates to pharmaceutical compositions comprising a therapeutically effective amount of a selective alpha-adrenergic receptor agonist or an anticholinergic agent and lipoic acid or a pharmaceutically acceptable salt or a stereoisomer thereof in combination with a therapeutically effective amount of lipoic acid or a pharmaceutically acceptable salt or a stereoisomer thereof.

The present disclosure relates to pharmaceutical compositions comprising a therapeutically effective amount of a selective alpha-adrenergic receptor agonist or an anticholinergic agent and lipoic acid or a pharmaceutically acceptable salt or a stereoisomer thereof in combination with a therapeutically effective amount of lipoic acid or a pharmaceutically acceptable salt or a stereoisomer thereof.

The present disclosure relates to pharmaceutical compositions comprising physical mixture of a therapeutically effective amount of a selective alpha-adrenergic receptor agonist or a pharmaceutically acceptable salt or a stereoisomer or a enantiomer thereof in combination with a therapeutically effective amount of lipoic acid or a pharmaceutically acceptable salt or a stereoisomer thereof.

The present disclosure relates to pharmaceutical compositions wherein, the selective alpha-adrenergic receptor agonist is selected from alpha 1-adrenergic receptor agonist or alpha 2-adrenergic receptor agonist or partial alpha 1-adrenergic receptor agonist and/or partial alpha 2-adrenergic receptor agonist.

The present disclosure relates to pharmaceutical compositions wherein, the lipoic acid is (R)-(+)-lipoic acid or (S)-(−)-lipoic acid or racemic mixture (R/S)-lipoic acid or preferably R-(+)-lipoic acid.

The present disclosure relates to pharmaceutical compositions wherein, the physical mixture comprises compound of Formula II is brimonidine tartrate and R-(+)-Lipoic acid.

The present disclosure relates to pharmaceutical compositions wherein, the physical mixture comprises compound of Formula III is oxymetazoline hydrochloride and R-(+)-Lipoic acid.

The disclosure also relates to a pharmaceutical composition of a therapeutically effective amount of a selective alpha-adrenergic receptor agonist or an anticholinergic agentor a pharmaceutically acceptable salt or a stereoisomer thereof in combination with a therapeutically effective amount of lipoic acid or a pharmaceutically acceptable salt or a stereoisomer thereof for use in the treatment or alleviation of xerostomia, burning mouth syndrome and eye diseases or disorders.

### DETAILED DESCRIPTION OF THE INVENTION

Disclosed herein are pharmaceutical compositions comprising a therapeutically effective amount of a selective alpha-adrenergic receptor agonist or an anticholinergic agent or a pharmaceutically acceptable salt or a stereoisomer thereof in combination with a therapeutically effective amount of lipoic acid or a pharmaceutically acceptable salt or a stereoisomer thereof.

In certain embodiments, the selective alpha-adrenergic receptor agonist or an anticholinergic agent is selected from the group consisting of pilocarpine, brimonidine, oxymetazoline, or a pharmaceutically acceptable salt or a stereoisomer thereof. In further embodiments, the antimuscarinic or anticholinergic agent is pilocarpine, or a pharmaceutically acceptable salt or a stereoisomer thereof.

In other embodiments, the selective alpha2-adrenergic receptor agonist is brimonidine, selective alpha1 and partial alpha2-adrenergic receptor agonist is oxymetazoline or a pharmaceutically acceptable salt or a stereoisomer thereof.

In certain embodiments, the antimuscarinic or anticholinergic agent is a compound of Formula (I):

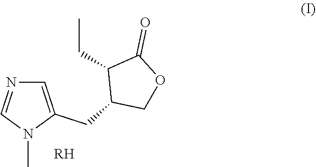

or a pharmaceutically acceptable salt or a stereoisomer thereof; wherein, RH is

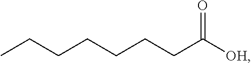

hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

In certain embodiments, the compositions are typically compounds in the forms of hydrates or solvates of pilocarpine and an acidic moiety RH containing compound selected RH in which the pilocarpine is protonated, and the acid moiety RH of the pharmaceutically acceptable salt is at least in partially ionic form. In some instances, however, for example depending on the pH of the environment, the composition may be in the form of a mixture of pilocarpine and acid components RH. In further embodiments, the compositions disclosed herein may further comprise a pharmaceutically acceptable carrier, diluent, or excipient, or a combination thereof.

In certain embodiments, the selective alpha2-adrenergic receptor agonist is a compound of Formula (II):

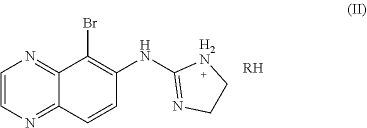

or a pharmaceutically acceptable salt or a stereoisomer thereof; wherein, RH is

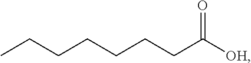

tartrate, hydrochloride, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

In certain embodiments, the compositions are typically compounds in the forms of salts of brimonidine and an acid moiety containing compound selected from RH in which the cevimeline is in protonated form and the acid moiety RH is at least in partially ionic form. In some instances, however, for example depending on the pH of the environment, the composition may be in the form of a mixture of brimonidine and an acid moiety RH. In further embodiments, the compositions disclosed herein may further comprise a pharmaceutically acceptable carrier, diluent, or excipient, or a combination thereof.

In certain embodiments, the selective alpha1 and partial alpha2-adrenergic receptor agonist is a compound of Formula (III):

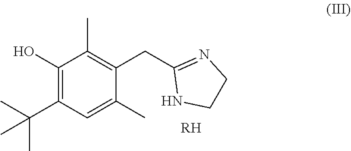

or a pharmaceutically acceptable salt or a stereoisomer thereof; wherein, RH is

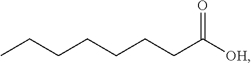

hydrochloric acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

In certain embodiments, the compositions are typically compounds in the forms of salts of oxymetazoline and an acid moiety containing compound selected from RH in which the oxymetazoline is in protonated form and the acid moiety RH is at least in partially ionic form. In some instances, however, for example depending on pH of the environment, the composition may be in the form of a mixture of oxymetazoline and an acid moiety RH. In further embodiments, the compositions disclosed herein may further comprise a pharmaceutically acceptable carrier, diluent, or excipient, or a combination thereof.

In certain embodiments, the lipoic acid choline ester prodrug is (R)-(+)-lipoic acid (RLA) or (S)-(−)-lipoic acid (SLA) or a racemic mixture (R/S)-lipoic acid (R/S-LA).

In certain embodiments, the lipoic acid choline ester prodrug is a compound of Formula (IV):

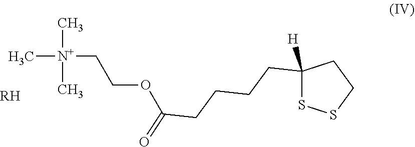

or a pharmaceutically acceptable salt or a stereoisomer thereof; wherein,
RH is H, NULL, glutamic acid, aspartic acid, lysine, ketorolac, ketoprofen, naproxen, bromine, diclofenac, nepafenac, bromfenac or glycine.

In certain embodiments, the lipoic acid is a compound of Formula (V):

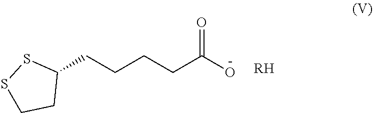

or a pharmaceutically acceptable salt or a stereoisomer thereof; wherein,
RH is H, NULL, sodium, potassium, magnesium, calcium, arginine, glutamate, lysine, glycine, proline, pyridoxine, pyridoxamine, choline, taurine, malic acid, PHMB, polyhexanide or guanidine.

It is to be contemplated that when a particular compound is mentioned by name, for example, pilocarpine, brimonidine, lipoic acid or oxymetazoline, the scope of the present disclosure encompasses pharmaceutically acceptable salts, esters, amides, or prodrugs of the named compound. Further, when the named compound comprises a chiral center the scope of the present disclosure also includes compositions comprising the racemic mixture of the two enantiomers, as well as compositions comprising each enantiomer individually substantially free of the other enantiomer. In further embodiments, if the named compound comprises more than one chiral center, the scope of the present disclosure also includes compositions comprising a mixture of the various diastereomers, as well as compositions comprising each diastereomer substantially free of the other diastereomers. Further, for example, commercially available pilocarpine comprises two stereocenters. The scope of the present disclosure includes pharmaceutical compositions comprising all four diastereomers, pharmaceutical compositions comprising the racemic mixture of R,R and S,S isomers, pharmaceutical compositions comprising the racemic mixture of R,S and S,R isomers, pharmaceutical compositions comprising the R,R enantiomer substantially free of the other diastereomers, pharmaceutical compositions comprising the S,S enantiomer substantially free of the other diastereomers, pharmaceutical compositions comprising the R,S enantiomer substantially free of the other diastereomers, and pharmaceutical compositions comprising the S,R enantiomer substantially free of the other diastereomers.

In certain embodiments, the composition comprising R enantiomer is substantially free of S enantiomer, or a composition comprising S enantiomer is substantially free of R enantiomer. In this context, “substantially free” means, the composition comprises less than about 20%, or less than about 15%, or less than about 10%, or less than about 5%, or less than about 3% or less than about 1% of the minor enantiomer.

In certain embodiments, the present disclosure relates to a pharmaceutical composition comprising pilocarpine in combination with lipoic acid prodrug. In further embodiments, the pharmaceutical composition comprises pilocarpine in combination with R-lipoic acid prodrug. In yet other embodiments, the present disclosure relates to a composition comprising a compound of Formula (I) in combination with a compound of Formula (IV). In further embodiments, the pharmaceutical composition comprises pilocarpine hydrochloride in combination with racemic lipoic acid prodrug. In other embodiments, the pharmaceutical composition comprises pilocarpine hydrochloride in combination with R-lipoic acid prodrug.

In certain embodiments, the present disclosure relates to a pharmaceutical composition comprising pilocarpine in combination with lipoic acid. In further embodiments, the pharmaceutical composition comprises pilocarpine in combination with R-lipoic acid. In yet other embodiments, the present disclosure relates to a composition comprising a compound of Formula (I) in combination with a compound of Formula (V). In further embodiments, the pharmaceutical composition comprises pilocarpine hydrochloride in combination with racemic lipoic acid. In other embodiments, the pharmaceutical composition comprises pilocarpine hydrochloride in combination with R-lipoic acid.

In certain embodiments, the present disclosure relates to a pharmaceutical composition comprising brimonidine in combination with lipoic acid prodrug. In further embodiments, the pharmaceutical composition comprises brimonidine in combination with R-lipoic acid prodrug. In yet other embodiments, the present disclosure relates to a composition comprising a compound of Formula (II) in combination with a compound of Formula (IV). In further embodiments, the pharmaceutical composition comprises brimonidine tartrate in combination with racemic lipoic acid prodrug. In other embodiments, the pharmaceutical composition comprises brimonidine tartrate in combination with R-lipoic acid prodrug.

In certain embodiments, the present disclosure relates to a pharmaceutical composition comprising brimonidine in combination with lipoic acid. In further embodiments, the pharmaceutical composition comprises brimonidine in combination with R-lipoic acid. In yet other embodiments, the present disclosure relates to a composition comprising a compound of Formula (II) in combination with a compound of Formula (V). In further embodiments, the pharmaceutical composition comprises brimonidine tartrate in combination with racemic lipoic acid. In other embodiments, the pharmaceutical composition comprises brimonidine tartrate in combination with R-lipoic acid.

In yet further embodiments, the present disclosure relates to a pharmaceutical composition comprising oxymetazoline in combination with lipoic acid prodrug. In further embodiments, the pharmaceutical composition comprises oxymetazoline in combination with R-lipoic acid prodrug. In yet other embodiments, the present disclosure relates to a composition comprising a compound of Formula (III) in combination with a compound of Formula (IV). In yet other embodiments, the pharmaceutical composition comprises oxymetazoline hydrochloride in combination with racemic lipoic acid prodrug. In other embodiments, the pharmaceutical composition comprises oxymetazoline hydrochloride in combination with R-lipoic acid prodrug.

In yet further embodiments, the present disclosure relates to a pharmaceutical composition comprising oxymetazoline in combination with lipoic acid. In further embodiments, the pharmaceutical composition comprises oxymetazoline in combination with R-lipoic acid. In yet other embodiments, the present disclosure relates to a composition comprising a compound of Formula (III) in combination with a compound of Formula (V). In yet other embodiments, the pharmaceutical composition comprises oxymetazoline hydrochloride in combination with racemic lipoic acid. In other embodiments, the pharmaceutical composition comprises oxymetazoline hydrochloride in combination with R-lipoic acid.

The compositions as disclosed herein can be used as a medicament. In certain embodiments, the compositions are particularly useful in treatment or alleviation of xerostomia, burning mouth syndrome and eye diseases or disorders. In certain embodiments, the compositions are useful in the treatment or alleviation of xerostomia or its related complications. The compositions for example, useful in treating a subject suffering from xerostomia or its related complications manifested from metabolic or genetic conditions or disorders, metabolic diseases, chronic diseases or disorders; cancer, dermal, skin, vascular or ocular complications.

In further embodiments, the compositions as disclosed herein are useful in the treatment or alleviation of burning mouth syndrome.

In further embodiments, the compositions as disclosed herein are useful in the treatment or alleviation of dermal diseases such as rosacea, psoriasis, allergy, flushing, itching or related dermal complications.

In further embodiments, the compositions as disclosed herein are useful in the treatment or alleviation of one or more eye diseases or disorders; wherein the eye disease or disorder is selected from the group consisting of presbyopia, retinal arterial occlusions, (in particular central retinal artery occlusion), age related visual degradation (near and far visual acuity; visual field), diabetic retinopathy, retinal vein occlusion (in particular central retinal vein occlusion or branch retinal vein occlusion), visual degradation of visual acuity and visual field, exsudative macular degeneration (age related macular degeneration, high myopia; macular degeneration), myopia, macular oedema, ocular redness, conjunctivitis, eye allergy, central serious chorio-retinopathy, papillitis, uveitis, glaucoma and/or glaucomatous neuropathy. In yet other embodiments, the compositions disclosed herein are useful in the treatment or alleviation of presbyopia, glaucoma and/or glaucomatous neuropathy.

In certain embodiments, the disclosure also provides a kit comprising any of the pharmaceutical compositions disclosed herein. The kit may comprise instructions for use in the treatment of xerostomia or its related complications, burning mouth syndrome or eye diseases or disorders.

In further embodiments, the present disclosure also relates to a method of treating xerostomia or eye disorders in a subject comprising administering to a subject in need thereof a therapeutically effective amount of an anticholinergic agentor a pharmaceutically acceptable salt or a stereoisomer thereof in combination with a therapeutically effective amount of lipoic acid prodrug or a pharmaceutically acceptable salt or a stereoisomer thereof; wherein the antimuscarinic or an anticholinergic agent and lipoic acid or lipoic acid prodrug are as described above. In certain embodiments, the subject is a mammal such as a human, or a non-human mammal. In further embodiments, the subject is a human.

In further embodiments, the present disclosure also relates to a method of treating eye disorders in a subject comprising administering to a subject in need thereof a therapeutically effective amount of an alpha-adrenergic receptor agonist or a pharmaceutically acceptable salt or a stereoisomer thereof in combination with a therapeutically effective amount of lipoic acid or lipoic acid prodrug or a pharmaceutically acceptable salt or a stereoisomer thereof; wherein the alpha2-adrenergic receptor agonist and lipoic acid or lipoic acid prodrug are as described above. In certain embodiments, the subject is a mammal such as a human, or a non-human mammal. In further embodiments, the subject is a human.

The present disclosure also relates to a method of treating one or more eye diseases or disorders in a subject comprising administering to a subject in need thereof a therapeutically effective amount of a selective alpha2-adrenergic receptor agonist or an anticholinergic agent or a pharmaceutically acceptable salt or a stereoisomer thereof in combination with a therapeutically effective amount of lipoic acid or lipoic acid prodrug or a pharmaceutically acceptable salt or a stereoisomer thereof; wherein the a selective alpha2-adrenergic receptor agonist or an anticholinergic agent and lipoic acid or lipoic acid prodrug are as described above, and the eye disease or disorder is selected from the group consisting of presbyopia, glaucoma, conjunctivitis, ocular redness, eye allergy, retinal arterial occlusions, (in particular central retinal artery occlusion), age related visual degradation (near and far visual acuity; visual field), diabetic retinopathy, retinal vein occlusion (in particular central retinal vein occlusion or branch retinal vein occlusion), visual degradation of visual acuity and visual field, exsudative macular degeneration (age related macular degeneration, high myopia; macular degeneration), myopia, macular oedema, central serious chorio-retinopathy, papillitis, uveitis, glaucoma and/or glaucomatous neuropathy. In further embodiments, the eye disease or disorder is presbyopia, glaucoma or glaucomatous neuropathy. In yet other embodiments, the subject is a mammal such as a human, or a non-human mammal. In further embodiments, the subject is a human.

In certain embodiments, a selective alpha2-adrenergic receptor agonist or an anticholinergic agent and lipoic acid or lipoic acid prodrug may be administered simultaneously or separately. In further embodiments, a selective alpha2-adrenergic receptor agonist or an anticholinergic agent may be administered prior to the lipoic acid or lipoic acid prodrug. In yet other embodiments, the antimuscarinic or an anticholinergic may be administered subsequent to the lipoic acid or lipoic acid prodrug. In some embodiments, when a selective alpha2-adrenergic receptor agonist or an anticholinergic agent and lipoic acid or lipoic acid prodrug may be administered within one dosage form. In further embodiments, when a selective alpha2-adrenergic receptor agonist or an anticholinergic agent and lipoic acid or lipoic acid prodrug may be administered within different dosage forms.

In certain embodiments, the alpha-adrenergic receptor agonist and lipoic acid or lipoic acid prodrug may be administered simultaneously or separately. In further embodiments, the alpha-adrenergic receptor agonist may be administered prior to the lipoic acid or lipoic acid prodrug. In yet other embodiments, the alpha-adrenergic receptor agonist may be administered subsequent to the lipoic acid or lipoic acid prodrug. In some embodiments, when the antimuscarinic or an alpha-adrenergic receptor agonist and lipoic acid or lipoic acid prodrug may be administered within one dosage form. In further embodiments, when the alpha-adrenergic receptor agonist and lipoic acid or lipoic acid prodrug may be administered within different dosage forms.

In certain embodiments of the compositions, a selective alpha2-adrenergic receptor agonist or an anticholinergic agent, or a pharmaceutically acceptable salt or a stereoisomer thereof, may present in a dose of from 0.01 mg to 50 mg. In further embodiments, a selective alpha2-adrenergic receptor agonist or an anticholinergic agent is present in a dose of 0.01 mg to 40 mg. In other embodiments, a selective alpha2-adrenergic receptor agonist or an anticholinergic agent or a pharmaceutically acceptable salt or a stereoisomer thereof, is present in a dose of 1 g to 20 g. In yet other embodiments, the a selective alpha2-adrenergic receptor agonist or an anticholinergic agent or a pharmaceutically acceptable salt or a stereoisomer thereof, is present in a dose of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg.

In certain embodiments of the compositions, the selective alpha2 adrenergic agonist, or a pharmaceutically acceptable salt or a stereoisomer thereof, may present in a dose from 0.0001 mg to 50 mg. In further embodiments, the selective alpha2 adrenergic agonist is present in a dose of 0.01 mg to 40 mg. In other embodiments, the selective alpha2 adrenergic agonist or a pharmaceutically acceptable salt or a stereoisomer thereof, is present in a dose of 0.01 g to 20 g. In yet other embodiments, the selective alpha2 adrenergic agonist or a pharmaceutically acceptable salt or a stereoisomer thereof, is present in a dose of 0.01 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg.

In certain embodiments of the compositions, the selective alpha 1 and partial alpha 2 adrenergic agonist, or a pharmaceutically acceptable salt or a stereoisomer thereof, may present in a dose of from 0.0001 mg to 50 mg. In further embodiments, the selective alpha1 and partial alpha 2 adrenergic agonist is present in a dose of 0.01 mg to 40 mg. In other embodiments, the selective alpha1 and partial alpha 2 adrenergic agonist or a pharmaceutically acceptable salt or a stereoisomer thereof, is present in a dose of 0.01 g to 20 g. In yet other embodiments, the selective alpha 1 and partial alpha 2 adrenergic agonist or a pharmaceutically acceptable salt or a stereoisomer thereof, is present in a dose of 0.01 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg.

In further embodiments, the lipoic acid prodrug or a pharmaceutically acceptable salt or a stereoisomer thereof, may present in a dose of from 0.1 mg to 4 g. In further embodiments, the lipoic acid prodrug or a pharmaceutically acceptable salt or a stereoisomer thereof is present in a dose of 1 mg, 5 mg, 10 mg, 20 mg to 2 g or 100 mg to 1.5 g. In yet other embodiment, the lipoic acid prodrug or a pharmaceutically acceptable salt or a stereoisomer thereof is present in a dose of 500 mg to 1 g.

In further embodiments, the lipoic acid or a pharmaceutically acceptable salt or a stereoisomer thereof, may present in a dose of from 0.05 mg to 4 g. In further embodiments, the lipoic acid or a pharmaceutically acceptable salt or a stereoisomer thereof is present in a dose of 0.10 mg to 2 g or 100 mg to 1.5 g. In yet other embodiment, the lipoic acid or a pharmaceutically acceptable salt or a stereoisomer thereof is present in a dose of 0.500 mg to 1 g.

The application also discloses a pharmaceutical composition comprising a pharmaceutically acceptable carrier and any of the compositions described herein. In certain embodiments, the pharmaceutical composition is formulated for ocular drop, ocular solution, eye drop, ocular insert, gel, ointment, ocular gel, ocular ointment, ocular paste, oral solution, oral rinsing solution, oral antiseptic solution, systemic administration, oral administration, oral mucoadhesvive spray, lozenge, buccal tablet, hard gelatin mouth dissolving tablet, effervescent tablet, mouth dissolving tablet, dermal, hydrogel, sustained release, parenteral administration, ocular, injection, dermal, topical, subdermal administration, or transdermal administration.

In certain embodiments, the active ingredients of the combination of the present disclosure can be administered by same or different route of administration. For example, the alpha adrenergic agonist or an anticholinergic agent of the present disclosure can be administered ocular, dermal, topical, orally, and the lipoic acid or lipoic acid prodrug can be administered dermal, ocular, topical, oral or transdermal.

In certain embodiments, the pharmaceutical compositions described herein are formulated in a manner such that said compositions will be delivered to a subject in a therapeutically effective amount, as part of a prophylactic or therapeutic treatment. The desired amount of the composition to be administered to a subject will depend on absorption, inactivation, and excretion rates of the drug as well as the delivery rate of the hydrates or solvates and compositions from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of any particular solvate or hydrate or composition may be adjusted to accommodate variations in the treatment parameters. Such treatment parameters include the clinical use to which the preparation is put, e.g., the site treated, the type of subject, e.g., human or non-human, adult or child, and the nature of the disease or condition.

In certain embodiments, the dosage of the subject compositions provided herein may be determined by reference to the plasma concentrations of the therapeutic composition or other encapsulated materials. For example, the maximum plasma concentration (Cmax) and the area under the plasma concentration-time curve from time 0 to infinity may be used. When used with respect to a pharmaceutical composition or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, mucus secretions, lymph or the like, one or more of the pharmaceutically acceptable excipients may undergo gradual or delayed degradation (e.g., through hydrolysis) with concomitant release of any material incorporated therein, e.g., a therapeutic and/or biologically active solvate or hydrate and/or composition, for a sustained or extended period (as compared to the release from a bolus). This release may result in prolonged delivery of therapeutically effective amounts of any of the therapeutic agents disclosed herein.

The phrases “systemic administration,” “administered systemically,” “peripheral administration” and “administered peripherally” are art-recognized, and include the administration of a subject composition, therapeutic or other material at a site remote from the disease being treated. Administration of an agent for the disease being treated, even if the agent is subsequently distributed systemically, may be termed “local” or “topical” or “regional” administration, other than directly into the central nervous system, e.g., by subcutaneous administration, such that it enters the subject's system and, thus, is subject to metabolism and other like processes.

The phrase “therapeutically effective amount” is an art-recognized term. In certain embodiments, the term refers to an amount of a solvate or hydrate or composition disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular composition without necessitating undue experimentation.

In certain embodiments, the pharmaceutical compositions described herein will incorporate the disclosed compounds and compositions to be delivered in an amount sufficient to deliver to a subject a therapeutically effective amount of a compound or composition as disclosed herein as part of a prophylactic or therapeutic treatment. The desired concentration of compounds as disclosed herein, or its pharmaceutical acceptable salts or stereoisomer thereof will depend on absorption, inactivation, and excretion rates of the drug as well as the delivery rate of the hydrates or solvates and compositions from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of any compound as disclosed herein may be adjusted to accommodate variations in the treatment parameters. Such treatment parameters include the clinical use to which the preparation is put, e.g., the site treated, the type of subject, e.g., human or non-human, adult or child, and the nature of the disease or condition.

The concentration and/or amount of any compound as disclosed herein may be readily identified by routine screening in animals, e.g., rats, by screening a range of concentration and/or amounts of the material in question using appropriate assays. Known methods are also available to assay local tissue concentrations, diffusion rates of the hydrates or solvates or compositions, and local blood flow before and after administration of therapeutic formulations disclosed herein. One such method is microdialysis, as reviewed by T. E. Robinson et al., 1991, microdialysis in the neurosciences, Techniques, volume 7, Chapter 1. The methods reviewed by Robinson may be applied, in brief, as follows. A microdialysis loop is placed in situ in a test animal. Dialysis fluid is pumped through the loop. When compounds such as those disclosed herein are injected adjacent to the loop, released drugs are collected in the dialysate in proportion to their local tissue concentrations. The progress of diffusion of the hydrates or solvates or compositions may be determined thereby with suitable calibration procedures using known concentrations of hydrates or solvates or compositions.

In certain embodiments, the dosage of the compounds disclosed herein may be determined by reference to the plasma concentrations of the therapeutic composition or other encapsulated materials. For example, the maximum plasma concentration (Cmax) and the area under the plasma concentration-time curve from time 0 to infinity may be used.

Generally, in carrying out the methods detailed in this application, an effective dosage for the compounds disclosed herein is in the range of about 0.001 mg/kg/day to about 100 mg/kg/day in single or divided doses, for instance 0.001 mg/kg/day to about 50 mg/kg/day in single or divided doses. The compounds may be administered at a dose of, for example, less than 0.2 mg/kg/day, 0.5 mg/kg/day, 1.0 mg/kg/day, 5 mg/kg/day, 10 mg/kg/day, 20 mg/kg/day, 30 mg/kg/day, or 40 mg/kg/day. Compounds may also be administered to a human subject at a dose of, for example, between 0.1 mg and 1000 mg, between 5 mg and 80 mg, or less than 1.0, 9.0, 12.0, 20.0, 50.0, 75.0, 100, 300, 400, 500, 800, 1000, 2000, 5000 mg per day. In certain embodiments, the compositions herein are administered at an amount that is less than 95%, 90%, 80%, 70%, 60%, 50%, 40%, 30%, 20%, or 10% of the compound as disclosed herein required for the same therapeutic benefit. In certain embodiments of the compositions, a selective alpha2-adrenergic receptor agonist or an anticholinergic agent, or a pharmaceutically acceptable salt or a stereoisomer thereof, may present in a dose of from 0.01 mg to 50 mg. In further embodiments, a selective alpha2-adrenergic receptor agonist or an anticholinergic agent is present in a dose of 0.01 mg to 40 mg. For example, a selective alpha2-adrenergic receptor agonist or an anticholinergic agent (such as pilocarpine, cevimeline, brimonidine, oxymetazoline and bethanechol) or a pharmaceutically acceptable salt or a stereoisomer thereof, is present in a dose of 1 g to 20 g. In yet other embodiments, the a selective alpha2-adrenergic receptor agonist or an anticholinergic agent or a pharmaceutically acceptable salt or a stereoisomer thereof, is present in a dose of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg.

The compositions provided by this application may be administered to a subject in need of treatment by a variety of conventional routes of administration, including orally, topically, parenterally, e.g., intravenously, subcutaneously or intramedullary. Further, the compositions may be administered intranasally, as a rectal suppository, or using a “flash” formulation, i.e., allowing the medication to dissolve in the mouth without the need to use water. Furthermore, the compositions may be administered to a subject in need of treatment by controlled release dosage forms, site specific drug delivery, transdermal drug delivery, patch (active/passive) mediated drug delivery, by stereotactic injection, or in nanoparticles.

The compositions may be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The pharmaceutical compositions formed by combining the compositions and the pharmaceutically acceptable carriers, vehicles or diluents are then readily administered in a variety of dosage forms such as tablets, powders, lozenges, sterile eye solution, ocular solution, syrups, injectable solutions and the like. These pharmaceutical compositions can, if desired, contain additional ingredients such as flavorings, binders, excipients and the like. Thus, for purposes of oral administration, tablets containing various excipients such as L-arginine, sodium citrate, calcium carbonate and calcium phosphate may be employed along with various disintegrates such as starch, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Appropriate materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof. The compounds as disclosed herein may also comprise enterically coated comprising of various excipients, as is well known in the pharmaceutical art.

For parenteral administration, solutions of the compositions may be prepared in (for example) sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

Generally, a composition as described herein may be administered orally, or parenterally (e.g., intravenous, intramuscular, subcutaneous or intramedullary). Topical administration may also be indicated, for example, where the subject is suffering from gastrointestinal disorder that prevent oral administration, or whenever the medication is best applied to the surface of a tissue or organ as determined by the attending physician. Localized administration may also be indicated, for example, when a high dose is desired at the target tissue or organ. For buccal administration the active composition may take the form of tablets or lozenges formulated in a conventional manner.

Illustratively, dosage levels of the administered active ingredients are: intravenous, 0.1 to about 200 mg/kg; intramuscular, 1 to about 500 mg/kg; orally, 5 to about 1000 mg/kg; intranasal instillation, 5 to about 1000 mg/kg; and aerosol, 5 to about 1000 mg/kg of host body weight.

Expressed in terms of concentration, an active ingredient can be present in the compositions of the present invention for localized use about the cutis, intranasally, pharyngolaryngeally, bronchially, intravaginally, rectally, or ocularly in a concentration of from about 0.01 to about 50% w/w of the composition; preferably about 1 to about 20% w/w of the composition; and for parenteral use in a concentration of from about 0.05 to about 50% w/v of the composition and preferably from about 5 to about 20% w/v.

The compositions of the present disclosure are preferably presented for administration to humans and animals in unit dosage forms, such as tablets, capsules, pills, powders, sterile ocular solution, sterile eye solution, ocular implant mediated delivery, granules, suppositories, sterile parenteral solutions or suspensions, sterile non-parenteral solutions of suspensions, and oral solutions or suspensions and the like, containing suitable quantities of an active ingredient. For oral administration either solid or fluid unit dosage forms can be prepared. For ocular administration either sterile solution or device or implant mediated delivery unit dosage forms can be prepared.

As discussed above, the tablet core contains one or more hydrophilic polymers. Suitable hydrophilic polymers include, but are not limited to, water swellable cellulose derivatives, polyalkylene glycols, thermoplastic polyalkylene oxides, acrylic polymers, hydrocolloids, clays, gelling starches, swelling cross-linked polymers, and mixtures thereof. Examples of suitable water swellable cellulose derivatives include, but are not limited to, sodium carboxymethylcellulose, cross-linked hydroxypropylcellulose, hydroxypropyl cellulose (HPC), hydroxypropylmethylcellulose (HPMC), hydroxyisopropylcellulose, hydroxybutylcellulose, hydroxyphenylcellulose, hydroxyethylcellulose (HEC), hydroxypentylcellulose, hydroxypropylethylcellulose, hydroxypropylbutylcellulose, and hydroxypropylethylcellulose, and mixtures thereof. Examples of suitable polyalkylene glycols include, but are not limited to, polyethylene glycol. Examples of suitable thermoplastic polyalkylene oxides include, but are not limited to, poly(ethylene oxide). Examples of suitable acrylic polymers include, but are not limited to, potassium methacrylatedivinylbenzene copolymer, polymethylmethacrylate, high-molecular weight crosslinked acrylic acid homopolymers and copolymers such as those commercially available from Noveon Chemicals under the tradename CARBOPOL™. Examples of suitable hydrocolloids include, but are not limited to, alginates, agar, guar gum, locust bean gum, kappa carrageenan, iota carrageenan, tara, gum arabic, tragacanth, pectin, xanthan gum, gellan gum, maltodextrin, galactomannan, pusstulan, laminarin, scleroglucan, gum arabic, inulin, pectin, gelatin, whelan, rhamsan, zooglan, methylan, chitin, cyclodextrin, chitosan, and mixtures thereof. Examples of suitable clays include, but are not limited to, smectites such as bentonite, kaolin, and laponite; magnesium trisilicate; magnesium aluminum silicate; and mixtures thereof. Examples of suitable gelling starches include, but are not limited to, acid hydrolyzed starches, swelling starches such as sodium starch glycolate and derivatives thereof, and mixtures thereof. Examples of suitable swelling cross-linked polymers include, but are not limited to, cross-linked polyvinyl pyrrolidone, cross-linked agar, and cross-linked carboxymethylcellulose sodium, and mixtures thereof.

The carrier may contain one or more suitable excipients for the formulation of tablets. Examples of suitable excipients include, but are not limited to, fillers, adsorbents, binders, disintegrants, lubricants, glidants, release-modifying excipients, superdisintegrants, antioxidants, and mixtures thereof.

Suitable binders include, but are not limited to, dry binders such as polyvinyl pyrrolidone and hydroxypropylmethylcellulose; wet binders such as water-soluble polymers, including hydrocolloids such as acacia, alginates, agar, guar gum, locust bean, carrageenan, carboxymethylcellulose, tara, gum arabic, tragacanth, pectin, xanthan, gellan, gelatin, maltodextrin, galactomannan, pusstulan, laminarin, scleroglucan, inulin, whelan, rhamsan, zooglan, methylan, chitin, cyclodextrin, chitosan, polyvinyl pyrrolidone, cellulosics, sucrose, and starches; and mixtures thereof. Suitable disintegrants include, but are not limited to, sodium starch glycolate, cross-linked polyvinylpyrrolidone, cross-linked carboxymethylcellulose, starches, microcrystalline cellulose, and mixtures thereof.

Suitable lubricants include, but are not limited to, long chain fatty acids and their hydrates or solvates, such as magnesium stearate and stearic acid, talc, glycerides waxes, and mixtures thereof. Suitable glidants include, but are not limited to, colloidal silicon dioxide. Suitable release-modifying excipients include, but are not limited to, insoluble edible materials, pH-dependent polymers, and mixtures thereof.

Suitable insoluble edible materials for use as release-modifying excipients include, but are not limited to, water-insoluble polymers and low-melting hydrophobic materials, copolymers thereof, and mixtures thereof. Examples of suitable water-insoluble polymers include, but are not limited to, ethylcellulose, polyvinyl alcohols, polyvinyl acetate, polycaprolactones, cellulose acetate and its derivatives, acrylates, methacrylates, acrylic acid copolymers, copolymers thereof, and mixtures thereof. Suitable low-melting hydrophobic materials include, but are not limited to, fats, fatty acid esters, phospholipids, waxes, and mixtures thereof. Examples of suitable fats include, but are not limited to, hydrogenated vegetable oils such as for example cocoa butter, hydrogenated palm kernel oil, hydrogenated cottonseed oil, hydrogenated sunflower oil, and hydrogenated soybean oil, free fatty acids and their hydrates or solvates, and mixtures thereof. Examples of suitable fatty acid esters include, but are not limited to, sucrose fatty acid esters, mono-, di-, and triglycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, stearoyl macrogol-32 glycerides, and mixtures thereof. Examples of suitable phospholipids include phosphotidyl choline, phosphotidyl serene, phosphotidyl enositol, phosphotidic acid, and mixtures thereof. Examples of suitable waxes include, but are not limited to, carnauba wax, spermaceti wax, beeswax, candelilla wax, shellac wax, microcrystalline wax, and paraffin wax; fat-containing mixtures such as chocolate, and mixtures thereof. Examples of super disintegrants include, but are not limited to, croscarmellose sodium, sodium starch glycolate and cross-linked povidone (crospovidone). In certain embodiments, the tablet core contains up to about 5 percent by weight of such super disintegrant.

Examples of antioxidants include, but are not limited to, tocopherols, ascorbic acid, sodium pyrosulfite, butylhydroxytoluene, butylated hydroxyanisole, edetic acid, and edetate hydrates or solvates, and mixtures thereof. Examples of preservatives include, but are not limited to, citric acid, tartaric acid, lactic acid, malic acid, acetic acid, benzoic acid, and sorbic acid, and mixtures thereof.

In certain embodiments, the immediate release coating has an average thickness of at least 50 microns, such as from about 50 microns to about 2500 microns; e.g., from about 250 microns to about 1000 microns. In embodiment, the immediate release coating is typically compressed at a density of more than about 0.9 g/cc, as measured by the weight and volume of that specific layer.

In certain embodiments, the immediate release coating contains a first portion and a second portion, wherein at least one of the portions contains the second pharmaceutically active agent. In certain embodiments, the portions contact each other at a center axis of the tablet. In certain embodiments, the first portion includes the first pharmaceutically active agent and the second portion includes the second pharmaceutically active agent.

In certain embodiments, the first portion contains the first pharmaceutically active agent and the second portion contains the second pharmaceutically active agent. In certain embodiments, one of the portions contains a third pharmaceutically active agent. In certain embodiments one of the portions contains a second immediate release portion of the same pharmaceutically active agent as that contained in the tablet core.

In certain embodiments, the outer coating portion is prepared as a dry blend of materials prior to addition to the coated tablet core. In another embodiment the outer coating portion is included of a dried granulation including the pharmaceutically active agent.

Formulations with different drug release mechanisms described above could be combined in a final dosage form containing single or multiple units. Examples of multiple units include multilayer tablets, capsules containing tablets, beads, or granules in a solid or liquid form. Typical, immediate release formulations include compressed tablets, gels, films, coatings, liquids and particles that can be encapsulated, for example, in a gelatin capsule. Many methods for preparing coatings, covering or incorporating drugs, are known in the art.

The immediate release dosage, unit of the dosage form, i.e., a tablet, a plurality of drug-containing beads, granules or particles, or an outer layer of a coated core dosage form, contains a therapeutically effective quantity of the active agent with conventional pharmaceutical excipients. The immediate release dosage unit may or may not be coated and may or may not be admixed with the delayed release dosage unit or units (as in an encapsulated mixture of immediate release drug-containing granules, particles or beads and delayed release drug-containing granules or beads).

Extended release formulations are generally prepared as diffusion or osmotic systems, for example, as described in “Remington—The Science and Practice of Pharmacy”, 20th. Ed., Lippincott Williams & Wilkins, Baltimore, Md., 2000). A diffusion system typically consists of one of two types of devices, reservoir and matrix, which are well known and described in die art. The matrix devices are generally prepared by compressing the drug with a slowly dissolving polymer carrier into a tablet form.

An immediate release portion can be added to the extended release system by means of either applying an immediate release layer on top of the extended release core; using coating or compression processes or in a multiple unit system such as a capsule containing extended and immediate release beads.

Delayed release dosage formulations are created by coating a solid dosage form with a film of a polymer which is insoluble in the acid environment of the stomach, but soluble in the neutral environment of small intestines. The delayed release dosage units can be prepared, for example, by coating a drug or a drug-containing composition with a selected coating material. The drug-containing composition may be a tablet for incorporation into a capsule, a tablet for use as an inner core in a “coated core” dosage form, or a plurality of drug-containing beads, particles or granules, for incorporation into either a tablet or capsule.

A pulsed release dosage form is one that mimics a multiple dosing profile without repeated dosing and typically allows at least a twofold reduction in dosing frequency as compared to the drug presented as a conventional dosage form (e.g., as a solution or prompt drug-releasing, conventional solid dosage form). A pulsed release profile is characterized by a time period of no release (lag time) or reduced release followed by rapid drug release.

Each dosage form contains a therapeutically effective amount of active agent. In certain embodiments of dosage forms that mimic a twice daily dosing profile, approximately 30 wt. % to 70 wt. %, preferably 40 wt. % to 60 wt. %, of the total amount of active agent in the dosage form is released in the initial pulse, and, correspondingly approximately 70 wt. % to 3.0 wt. %, preferably 60 wt. % to 40 wt. %, of the total amount of active agent in the dosage form is released in the second pulse. For dosage forms mimicking the twice daily dosing profile, the second pulse is preferably released approximately 3 hours to less than 14 hours, and more preferably approximately 5 hours to 12 hours, following administration.

Another dosage form contains a compressed tablet or a capsule having a drug-containing immediate release dosage unit, a delayed release dosage unit and an optional second delayed release dosage unit. In this dosage form, the immediate release dosage unit contains a plurality of beads, granules particles that release drug substantially immediately following oral administration to provide an initial dose. The delayed release dosage unit contains a plurality of coated beads or granules, which release drug approximately 3 hours to 14 hours following oral administration to provide a second dose.

For purposes of transdermal (e.g., topical) administration, dilute sterile, aqueous or partially aqueous solutions (usually in about 0.1% to 5% concentration), otherwise similar to the above parenteral solutions, may be prepared.

Methods of preparing various pharmaceutical compositions with a certain amount of one or more compounds of Formula I, Formula II, Formula III or Formula IV other active agents are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

In addition, in certain embodiments, subject compositions of the present application maybe lyophilized or subjected to another appropriate drying technique such as spray drying. The subject compositions may be administered once or may be divided into a number of smaller doses to be administered at varying intervals of time, depending in part on the release rate of the compositions and the desired dosage.

Formulations useful in the methods provided herein include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of a subject composition which may be combined with a carrier material to produce a single dose may vary depending upon the subject being treated, and the particular mode of administration.

Methods of preparing these formulations or compositions include the step of bringing into association subject compositions with the carrier and, optionally, one or more accessory ingredients. In general, the formulations are prepared by uniformly and intimately bringing into association a subject composition with liquid carriers, or finely divided solid carriers, or both, and then, if necessary, shaping the product.

The compounds of Formula I, Formula II, Formula III, Formula IV or Formula V described herein may be administered in inhalant or aerosol formulations. The inhalant or aerosol formulations may comprise one or more agents, such as adjuvants, diagnostic agents, imaging agents, or therapeutic agents useful in inhalation therapy. The final aerosol formulation may for example contain 0.005-90% w/w, for instance 0.005-50%, 0.005-5% w/w, or 0.01-1.0% w/w, of medicament relative to the total weight of the formulation.

In solid dosage forms for oral administration (capsules, tablets, pills, dragees, powders, granules and the like), the subject composition is mixed with one or more pharmaceutically acceptable carriers and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using lactose or milk sugars, as well as high molecular weight polyethylene glycols and the like.

Liquid dosage forms for oral administration include pharmaceutically acceptable emulsions, microemulsions, solutions, suspensions, syrups and elixirs. In addition to the subject compositions, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, corn, peanut, sunflower, soybean, olive, castor, and sesame oils), glycerol, tetrahydrofuryl alcohol, polyethylene glycols and fatty acid esters of sorbitan, and mixtures thereof.

Suspensions, in addition to the subject compositions, may contain suspending agents such as, for example, ethoxylated isostearyl alcohols, polyoxyethylene sorbitol, and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating carriers comprising, for example, cocoa butter, polyethylene glycol, a suppository wax, or a salicylate, and which is solid at room temperature, but liquid at body temperature and, therefore, will melt in the appropriate body cavity and release the encapsulated compound(s) and composition(s). Formulations which are suitable for vaginal administration also include pessaries, tampons, creams, gels, pastes, foams, or spray formulations containing such carriers as are known in the art to be appropriate.

Dosage forms for transdermal administration include powders, sprays, ointments, pastes, creams, lotions, gels, solutions, patches, and inhalants. A subject composition may be mixed under sterile conditions with a pharmaceutically acceptable carrier, and with any preservatives, buffers, or propellants that may be required. For transdermal administration, the complexes may include lipophilic and hydrophilic groups to achieve the desired water solubility and transport properties.

The ointments, pastes, creams and gels may contain, in addition to subject compositions, other carriers, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays may contain, in addition to a subject composition, excipients such as lactose, talc, silicic acid, aluminum hydroxide, calcium silicates and polyamide powder, or mixtures of such substances. Sprays may additionally contain customary propellants, such as chlorofluorohydrocarbons and volatile unsubstituted hydrocarbons, such as butane and propane.

Methods of delivering a composition or compositions via a transdermal patch are known in the art. Exemplary patches and methods of patch delivery are described in U.S. Pat. Nos. 6,974,588, 6,564,093, 6,312,716, 6,440,454, 6,267,983, 6,239,180, and 6,103,275.

In another embodiment, a transdermal patch may comprise: a substrate sheet comprising a composite film formed of a resin composition comprising 100 parts by weight of a polyvinyl chloride-polyurethane composite and 2-10 parts by weight of a styrene-ethylene-butylene-styrene copolymer, a first adhesive layer on the one side of the composite film, and a polyalkylene terephthalate film adhered to the one side of the composite film by means of the first adhesive layer, a primer layer which comprises a saturated polyester resin and is formed on the surface of the polyalkylene terephthalate film; and a second adhesive layer comprising a styrene-diene-styrene block copolymer containing a pharmaceutical agent layered on the primer layer. A method for the manufacture of the above-mentioned substrate sheet comprises preparing the above resin composition molding the resin composition into a composite film by a calendar process, and then adhering a polyalkylene terephthalate film on one side of the composite film by means of an adhesive layer thereby forming the substrate sheet and forming a primer layer comprising a saturated polyester resin on the outer surface of the polyalkylene terephthalate film.

Another type of patch comprises incorporating the drug directly in a pharmaceutically acceptable adhesive and laminating the drug-containing adhesive onto a suitable backing member, e.g. a polyester backing membrane. The drug should be present at a concentration which will not affect the adhesive properties, and at the same time deliver the required clinical dose.

Transdermal patches may be passive or active. Passive transdermal drug delivery systems currently available, such as the nicotine, estrogen and nitroglycerine patches, deliver small-molecule drugs. Many of the newly developed proteins and peptide drugs are too large to be delivered through passive transdermal patches and may be delivered using technology such as electrical assist (iontophoresis) for large-molecule drugs.

Iontophoresis is a technique employed for enhancing the flux of ionized substances through membranes by application of electric current. One example of an iontophoretic membrane is given in U.S. Pat. No. 5,080,646. The principal mechanisms by which iontophoresis enhances molecular transport across the skin are (a) repelling a charged ion from an electrode of the same charge, (b) electroosmosis, the convective movement of solvent that occurs through a charged pore in response the preferential passage of counter-ions when an electric field is applied or (c) increase skin permeability due to application of electrical current.

Certain ranges are disclosed herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning and the meaning of such terms is independent at each occurrence thereof and is as commonly understood by one of skill in art to which the subject matter herein belongs.

The singular forms “a”, “an” and “the” encompass plural references unless the context clearly indicates otherwise.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, lngold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

As used herein, the term “metabolic condition” refers to an inborn error of metabolism (or genetic metabolic conditions) are genetic disorders that result from a defect in one or more metabolic pathways; specifically, the function of an enzyme is affected and is either deficient or completely absent.

The term “polymorph” as used herein is art-recognized and refers to one crystal structure of a given compound.

The phrases “parenteral administration” and “administered parenterally” as used herein refer to modes of administration other than enteral and topical administration, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intra-articular, subcapsular, subarachnoid, intraspinal and intrastemal injection and infusion.

The phrase “pharmaceutically acceptable” is art-recognized. In certain embodiments, the term includes compositions, polymers and other materials and/or dosage forms which are, within the scope of sound medical judgment, suitable for use in contact with the tissues of mammals, human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “prodrug” is intended to encompass compounds that, under physiological conditions, are converted into the therapeutically active agents of the present invention. A common method for making a prodrug is to include selected moieties that are hydrolyzed under physiological conditions to reveal the desired molecule. In other embodiments, the prodrug is converted by an enzymatic activity of the host animal.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic, (i.e., it is intended to diminish, ameliorate, or stabilize the existing unwanted condition or side effects thereof).

The term “predicting” as used herein refers to assessing the probability related diseases patient will suffer from abnormalities or complication and/or terminal platelet aggregation or failure and/or death (i.e. mortality) within a defined time window (predictive window) in the future. The mortality may be caused by the central nervous system or complication. The predictive window is an interval in which the subject will develop one or more of the said complications according to the predicted probability. The predictive window may be the entire remaining lifespan of the subject upon analysis by the method of the present invention.

As used herein, the term “stereoisomer” is a term used for all isomers of individual compounds of Formula (I), Formula (II), Formula (III), Formula (IV) or Formula (V) that differs only in the orientation of their atoms in space. The term stereoisomer includes minor image isomers (enantiomers) of compounds of Formula (I), Formula (II), Formula (III), Formula (IV) or Formula (V), mixtures of mirror image isomers (racemates, racemic mixtures) of compounds of Formula (I), Formula (II), Formula (III), Formula (IV) or Formula (V) geometric (cis/trans or E/Z, R/S) isomers of compounds of Formula (I), Formula (II), Formula (III) Formula (IV) or Formula (V) and isomers of compounds of Formula (I), Formula (II), Formula (III) Formula (IV) or Formula (V) with more than one chiral center that are not mirror images of one another (diastereoisomers).

As used herein, the term “subject,” that is interchangeable with “patient” or “host”, refers to an animal, preferably a mammal, and most preferably a human Subjects include primates and other mammals such as equines, cattle, swine and sheep; and poultry and pets in general.

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating xerostomia, dry mouth, dry mouth in Sjögren's syndrome, bladder and gastrointestinal problems and other related diseases or any other medical condition, is well understood in the art, and includes administration of a composition which reduces the frequency of, or delays the onset of, symptoms of a medical condition in a subject relative to a subject which does not receive the composition.

The phrase “therapeutically effective amount” is an art-recognized term. In certain embodiments, the term refers to an amount of a solvate or hydrate or composition disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular composition without necessitating undue experimentation.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present disclosure include such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present disclosure.

### METHOD OF SYNTHESIS

### Example: 1

Brimonidine-(R)-lipoate (CLX-SYN-G162-C02) is prepared by treating a solution of R-Lipoic acid in isopropanol with brimonidine free base (prepared from Brimonidine tartrate) at 25-30° C./2 hrs followed by filtration of solid which was dried at 40-45° C. for 4-5 hrs. Salt formation was confirmed by disappearance of carboxylic acid proton of lipoic acid (appearing at 12 ppm) in NMR of CLX-SYN-G162-C02 when performed in CDCl3.

Brimonidine free base and R-Lipoic acid have sharp melting points (MP of brimonidine 255-257° C. and R-lipoic acid 48-51° C.) while CLX-SYN-G162-C02 being a salt does not have a sharp melting point (142-226° C.) as detailed below in the table 1:

TABLE 1

| | | Melting point | |
|---|---|---|---|
| | | By Capillary melting | |
| S. No. | KSM/Reagent | point analysis | SOR |
| 1 | Brimonidine tartrate | 204-207° C. | +9° |
| 2 | R-Lipoic acid | 48-51° C. | 118° |
| 3 | Brimonidine Free base | 255-257° C. | Not optically active |
| 4 | (CLX-SYN-G162-C02) | 150-232° C. | +48.4° |
| | | 142-226° C. | +50° |

Brimonidine tartrate and R-Lipoic acid were mixed physically in equimolar ratio. Physicochemical properties of this residue (as detailed in the table 2 below) were studied which further confirm that no salt formation has taken place on physical mixing of brimonidine tartrate and R-Lipoic acid.

TABLE 2

| | KSM/Reagent/ | | | |
|---|---|---|---|---|
| S. No | Compound | Test | Result | Remarks |
| 1 | Brimonidine tartrate | Melting Point | 51-54° C. | Partial melting at 51-54° C. |
| | and R-Lipoic acid | (by Capillary | and | indicates the presence of |
| | equimolar mixture | MP analysis) | 190-196° C. | Free Lipoic acid and |
| | | | | complete melting 190-196° C. |
| | | | | with decomposition corresponds |
| | | | | to brimonidine tartrate. |
| | | | | A slight decrease in MP of |
| | | | | brimonidine tartrate in this |
| | | | | residue is due to the presence |
| | | | | of Lipoic acid as impurity. |
| | | DSC | 50.7° C. | Presence of two endotherms |
| | | | (Peak temp) & | (50.7° C. and 196.7° C.) are |
| | | | 196.7° C. | indicative of the presence of |
| | | | (Peak temp) | Free Lipoic acid and Free |
| | | | | brominidine tartrate in the |
| | | | | residue. This further supports |
| | | | | that CLX-SYN-G162-C02 |
| | | | | is not formed on physical mixing |
| | | | | of brimonidine tartrate and |
| | | | | R-Lipoic acid. |
| | | SOR | +40° | SOR is concentration dependent. |
| | | | | A drop in SOR is due to mixing |
| | | | | of two compounds having |
| | | | | different SOR. |
| 2 | CLX-SYN- | Melting | 142-226° C. | Broad range of melting but no |
| | G162C-02 | Point | | decomposition observed |
| | | SOR | +50° | — |

### Example 2

Oxymetazoline HCl and R-Lipoic acid were mixed physically in equimolar ratio. Physicochemical properties of this residue (as detailed in the table 3 & 4 below) were studied which further confirm that no salt formation has taken place on physical mixing of oxymatazoline HCl and R-Lipoic acid.

TABLE 3
Characterization of Oxymatazoline
HCl-R-Lipoic acid physical mixture:

| Model | P-1030 (A053560638) | |
|---|---|---|
| Room Temp. | 25 | Degree |
| Weight | 1.0034 | [%] |
| Sample | oxymatazoline HC1-R-lipoic acid 1% | |
| Comment | Methanol | |
| Mode | Specific Optical Rotation | |
| Light | Na | |
| Wavelength | 589 | nm |
| Cell path | 100.00 | mm |
| Concentration | 1.0034 | w/v% |
| Factor | 1.0000 | |
| Blank | 0.0000 | degree |
| Interval | 5 | sec |
| Integration | 5 | sec |

| | |
|---|---|
| Average | 59.0034 |
| S.D | 0.3251 |
| C.V. | 05510% |

TABLE 4
SOR readings Oxymatazoline HCl-R-Lipoic acid physical mixture:

| No | Sample No | Data | Temp |
|---|---|---|---|
| | 1 (1/5) | 58.900 | 25.2 |
| | 1 (2/5) | 59.577 | 25.1 |
| | 1 (3/5) | 58.920 | 24.9 |
| | 1 (4/5) | 58.830 | 24.8 |
| | 1 (5/5) | 58.790 | 24.7 |

### Example 3: Preclinical Study Report

Single Dose Comparative Ocular Pharmacokinetic Study of Brimonidine-(R)-Lipoate Formulation with Brimonidine Tartrate in Male New Zealand White Rabbits

The purpose of this study was to compare the ocular pharmacokinetics of Brimonidine-(R)-Lipoate (CLX-SYN-G162-C02)-Test compound and Brimonidine Tartrate-Reference compound when administered to male New Zealand white rabbits by topical ocular route.

Materials and Methods

### Test System:

TABLE 6

| | |
|---|---|
| Species | Rabbit |
| Strain | New Zealand White |
| Source | Liveon Biolabs Pvt. Ltd. |
| | Plot No. 46 and 47, Water Tank Road, |
| | II Phase, KIADB Industrial Area, |
| | Antharasanahalli, KasabaHobli, Karnataka |
| Total number of Rabbits | 24 Males (21 for ocular PK + 3 as controls) |
| Age at the treatment | Young healthy adults males of about 4-6 months |
| | old |
| Weight range at the start | ~ 2.0 to 3.0 Kg |
| of treatment | |
| Veterinary examination | Prior to the final assignment to the study, |
| | rabbits were subjected to a veterinary |
| | examination to ensure that the selected rabbits |
| | are in a good state of health. |
| Identification | Cage card and individual animal number on ear |
| | pinna using permanent ink marker. |
| Acclimatization | The rabbits were acclimatized for 5 days in the |
| | experimental room after veterinary examination. |
| | Both eyes of each animal provisionally selected |
| | for testing was examined within 24 hours |
| | before testing starts. Selected animals had no |
| | preexisting eye abnormalities. |

### Test Item Details

TABLE 7
Test Formulation (CLX-162)

| | |
|---|---|
| Test formulation code | CLX-162-TF2 |
| Name | 0.17% of CLX-SYN-G162-C02 |
| Batch Number | CLX162-PF00015-011-F1 |
| Storage condition | 15-25° C. |
| Molecular weight of | 498.46 |
| CLX-SYN-G162-C02 | |
| Molecular weight of | 292.135 |
| Brimonidine | |

Note:
1.7 mg/mL of CLX-SYN-G162-C02 is expected to deliver to 0.99 mg/mL of brimonidine and 0.7 mg/mL of (R)-Lipoic acid.

TABLE 8
Reference Formulation

| | |
|---|---|
| Name | 0.15% of brimonidine tartrate |
| Batch number | 98363 |
| Expiry date | April 2020 |
| Manufactured by | Allergan Inc., USA |
| Storage condition | 15-25° C. |

Note:
1.5 mg/mL of brimonidine tartrate is equivalent to 0.99 mg/mL of brimonidine.

### Composition of Test Formulations (CLX-162)

TABLE 9
The composition of CLX-162 ophthalmic solution is as below:

| Test Formulation | CLX-162-TF2 | |
|---|---|---|
| code | | |
| Dose | 1.7 mg/mL equivalent to 0.99 mg/mL of brimonidine | |
| concentration | and 0.7 mg/mL of (R)-Lipoic acid. | |
| Composition | 1. | CLX-162-TF2: 1.7 mg/ml. |
| | 2. | Sodium Carboxymethyl Cellulose (NaCMC)-5 |
| | | mg/mL |
| | 3. | Polysorbate 80-5 mg/mL |
| | 4. | Stabilized oxychloro complex (SOC)-0.05 mg/mL |
| | 5. | Sodium chloride-5.8 mg/mL |
| | 6. | Potassium chloride-1.40 mg/mL |
| | 7. | Calcium chloride dehydrate-0.2 mg/mL |
| | 8. | Magnesium chloride hexahydrate-0.06 mg/mL |
| | 9. | Boric acid-2 mg/mL |
| | 10. | Sodium borate decahydrate-~0.10-0.2 mg/ml to |
| | | adjust pH |
| | 11. | Water for Injection-q.s. |

### Study Design and Treatment:

### Treatment Method

A volume of 0.05 mL (50 μL) of the formulation as indicated in table 10 below was instilled topically into the inferior cul-de-sac of the each eye (both eyes; total=100 μL per rabbit) using a calibrated micropipette fitted with pre-sterilized micro tip and the eyes were closed for a minute after instillation.

TABLE 10

| Rabbit | Time point | | | |
|---|---|---|---|---|
| No. | for sampling | | Period 1 | Period 2 |
| 1-3 | 0.25 | h | Test | Reference |
| | | | Formulation | formulation |
| 4-6 | 0.5 | h | Test | Reference |
| | | | Formulation | formulation |
| 7-9 | 1.0 | h | Test | Reference |
| | | | Formulation | formulation |
| 10-12 | 2.0 | h | Test | Reference |
| | | | Formulation | formulation |
| 13-15 | 4.0 | h | Test | Reference |
| | | | Formulation | formulation |
| 16-18 | 8.0 | h | Test | Reference |
| | | | Formulation | formulation |
| 19-21 | 12.0 | h | Test | Reference |

| | |
|---|---|
| Formulation | formulation |

Animals were used in period 2 after a washout period of 6 days. N=3 rabbits were used for each time point.

In Vivo Sampling
One minute prior to sampling, corneas were locally anesthetized with 2% lignocaine (˜30 μL).
Aqueous humor (˜100 μL from each eye/time point) was collected with a 30-G sterile hypodermic needle via paracentesis at 0.25, 0.5, 1, 2, 4, 8 and 12 h from both eyes. N=3 rabbits were used for each time point (as specified in section 3.2).
Aqueous humor sample was transferred to a pre-labeled vial and were immediately snap-frozen in liquid nitrogen prior to storage on dry-ice during study.
The collected aqueous humor samples were stored at −70° C. until analysis.
Study was carried out in cross-over manner after washout period of 7 days (Period 1: Test formulation and Period 2: Reference compound).

### Pharmacokinetic Data Analysis and Evaluation

Pharmacokinetic parameters: Cₘₐₓ, Tₘₐₓ, T_{1/2}, AUC and MRT values were calculated using Phoenix® Software, version 8.0, USA. Additionally, concentrations of brimonidine in aqueous humor of animals treated with test formulations were compared against concentrations after treatment of reference formulation using two way ANOVA followed by Bonferroni's multiple comparison test (GraphPad Prism, Version 7.04).

### Results

Table 1: Mean brimonidine concentrations in aqueous humor of
New Zealand white rabbits.

TABLE 11
Brimonidine concentrations (ng/mL) in aqueous humor

| | Test formulation | | Reference formulation | |
|---|---|---|---|---|
| Time (h) | Mean | S.D. | Mean | S.D. |
| 0.25 | 695 | 236 | 466 | 91 |
| 0.50 | 1073 | 211 | 1008 | 171 |
| 1.00 | 813 | 244 | 518 | 248 |
| 2.00 | 415 | 114 | 343 | 74 |
| 4.00 | 66 | 56 | 66 | 37 |
| 8.00 | 0 | 0 | 0 | 0 |
| 12.00 | 0 | 0 | 0 | 0 |

Bonferroni's multiple comparison test: p values were greater than
0.05 (p>0.05) for Test formulation Vs Reference formulation.
Table 2 Brimonidine Pharmacokinetic parameters in aqueous humor of New Zealand white rabbits.

TABLE 12
Brimonidine in aqueous humor

| | Test | Reference | Test/ |
|---|---|---|---|
| Parameters | formulation | formulation | Reference |
| Cmax (ng/mL) | 1073 | 1008 | 1.06 |
| Tmax (h) | 0.50 | 0.50 | NA |
| T1/2 (h) | 0.82 | 0.98 | NA |
| MRTlast (h) | 1.26 | 1.30 | NA |
| AUClast (ng*h/mL) | 1875 | 1462 | 1.28 |
| AUCinf (ng*h/mL) | 1953 | 1555 | 1.26 |

N=3 animals (6 eyes)/time point/group

Result of the study indicates brimonidine levels were detectable in the aqueous humor up to 4 hour post dose in both test formulation and Reference formulation. Brimonidine was rapidly absorbed from both formulations with maximal aqueous humor concentrations being reached within 0.5 hour. The maximum aqueous humor concentration (Cmax) of brimonidine in Test Formulation and Reference formulation is comparable. The AUC of Test formulation is comparatively better than the Reference formulation.

What is claimed is:

1. A pharmaceutical compositions comprising physical mixture of:
a. a therapeutically effective amount of a selective alpha-adrenergic receptor agonist, or a pharmaceutically acceptable salt, a stereoisomer or an enantiomer thereof; and
b. a therapeutically effective amount of lipoic acid, or a pharmaceutically acceptable salt, stereoisomer or an enantiomer thereof.

2. The pharmaceutical composition of claim 1, wherein the selective alpha-adrenergic receptor agonist agent is selected from a compound of Formula II: 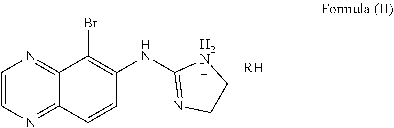
or a pharmaceutically acceptable salt or a stereoisomer thereof; and
wherein,
RH is 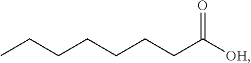
tartrate, hydrochloride, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine, furoate, methyl furoate, ethyl furoate, aminocaproic acid, caproic acid, caprilic acid, capric acid, lauric acid, alpha lipoic acid, R-lipoic acid, myristic acid, myristoleic acid, palmitic acid, palmitoleic acid, stearic acid, oleic acid, elaidic acid, linoleic acid, linolenic acid, linolelaidic acid or arachidonic acid.

3. The pharmaceutical composition of claim 1, wherein the selective alpha-adrenergic receptor agonist agent is selected from a compound of Formula (III): 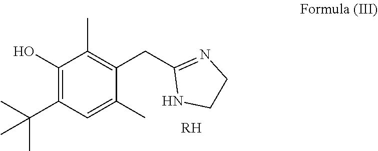
or a pharmaceutically acceptable salt or a stereoisomer thereof; and
wherein,
RH is as defined in claim 2.

4. The pharmaceutical composition of claim 1, wherein the lipoic acid is (R)-(+)-lipoic acid, (S)-(−)-lipoic acid or racemic mixture (R/S)-lipoic.

5. The pharmaceutical composition of claim 1, wherein the physical mixture comprises compound of Formula II is brimonidine tartrate and R-(+)-lipoic acid.

6. The pharmaceutical composition of claim 1, wherein the physical mixture comprises compound of Formula III is oxymetazoline hydrochloride and R-(+)-lipoic acid.

7. (canceled)

8. (canceled)

9. (canceled)

10. The pharmaceutical composition of claim 1, wherein the pharmaceutical composition further comprises at least one pharmaceutically acceptable excipient.

11. The pharmaceutical compositions as claimed in claim 10, wherein the pharmaceutical composition is formulated as drops, a solution, an emulsion, a paste, a gel, a cream, an ointment, a spray, tablets, effervescent tablets, a mucoadhesive formulation, a subdermal formulation, a transdermal formulation, a hydrogel, injections, or a sustained release formulation for oral, ocular, dermal, parenteral, subdermal, topical and nasal administration.

12. A method of using the pharmaceutical composition of claim 11, for the treatment or alleviation of xerostomia, burning mouth syndrome, dermal disorders and eye diseases or disorders or a complication thereof.

13. The pharmaceutical composition of claim 2, wherein the pharmaceutical composition further comprises at least one pharmaceutically acceptable excipient.

14. The pharmaceutical compositions as claimed in claim 13, wherein the pharmaceutical composition is formulated as drops, a solution, an emulsion, a paste, a gel, a cream, an ointment, a spray, tablets, effervescent tablets, a mucoadhesive formulation, a subdermal formulation, a transdermal formulation, a hydrogel, injections, or a sustained release formulation for oral, ocular, dermal, parenteral, subdermal, topical and nasal administration.

15. A method of using the pharmaceutical composition of claim 14, for the treatment or alleviation of xerostomia, burning mouth syndrome, dermal disorders and eye diseases or disorders or a complication thereof.

16. The pharmaceutical composition of claim 3, wherein the pharmaceutical composition further comprises at least one pharmaceutically acceptable excipient.

17. The pharmaceutical compositions as claimed in claim 16, wherein the pharmaceutical composition is formulated as drops, a solution, an emulsion, a paste, a gel, a cream, an ointment, a spray, tablets, effervescent tablets, a mucoadhesive formulation, a subdermal formulation, a transdermal formulation, a hydrogel, injections, or a sustained release formulation for oral, ocular, dermal, parenteral, subdermal, topical and nasal administration.

18. A method of using the pharmaceutical composition of claim 17, for the treatment or alleviation of xerostomia, burning mouth syndrome, dermal disorders and eye diseases or disorders or a complication thereof.

19. The pharmaceutical composition of claim 5, wherein the pharmaceutical composition further comprises at least one pharmaceutically acceptable excipient.

20. The pharmaceutical compositions as claimed in claim 19, wherein the pharmaceutical composition is formulated as drops, a solution, an emulsion, a paste, a gel, a cream, an ointment, a spray, tablets, effervescent tablets, a mucoadhesive formulation, a subdermal formulation, a transdermal formulation, a hydrogel, injections, or a sustained release formulation for oral, ocular, dermal, parenteral, subdermal, topical and nasal administration.

21. A method of using the pharmaceutical composition of claim 20, for the treatment or alleviation of xerostomia, burning mouth syndrome, dermal disorders and eye diseases or disorders or a complication thereof.

22. The pharmaceutical composition of claim 6, wherein the pharmaceutical composition further comprises at least one pharmaceutically acceptable excipient.

23. The pharmaceutical compositions as claimed in claim 22, wherein the pharmaceutical composition is formulated as drops, a solution, an emulsion, a paste, a gel, a cream, an ointment, a spray, tablets, effervescent tablets, a mucoadhesive formulation, a subdermal formulation, a transdermal formulation, a hydrogel, injections, or a sustained release formulation for oral, ocular, dermal, parenteral, subdermal, topical and nasal administration.

24. A method of using the pharmaceutical composition of claim 23, for the treatment or alleviation of xerostomia, burning mouth syndrome, dermal disorders and eye diseases or disorders or a complication thereof.
